(19) United States
(12) Patent Application Publication
(10) Pub. No.: US 2017/0325668 A1
(43) Pub. Date: Nov. 16, 2017

(54) SCANNING ENDOSCOPE DEVICE AND METHOD FOR CONTROLLING THE SAME

(71) Applicant: OLYMPUS CORPORATION, Tokyo (JP)

(72) Inventor: Keiichiro NAKAJIMA, Tokyo (JP)

(73) Assignee: OLYMPUS CORPORATION, Tokyo (JP)

(21) Appl. No.: 15/665,500

(22) Filed: Aug. 1, 2017

Related U.S. Application Data
(63) Continuation of application No. PCT/JP2015/000612, filed on Feb. 10, 2015.

(51) Int. Cl. A61B 1/00; G02B 23/24; A61B 1/06; A61B 1/07; G02B 23/26

(52) U.S. Cl. CPC A61B 1/00172; G02B 23/26; G02B 23/24; A61B 1/00096; A61B 1/0653; A61B 1/07; A61B 1/0017

(57) ABSTRACT

Provided is a scanning endoscope apparatus capable of generating an image having an optimum SNR. The scanning endoscope apparatus includes: a light source; an optical fiber that guides light emitted from the light source; an actuator that deflects light emitted from the optical fiber and repeatedly scans the deflected light on the irradiation object; a light detector with a controllable multiplication factor, the light detector photoelectrically converting signal light obtained from the irradiation object irradiated with the light; and a controller, the controller controlling the multiplication factor so as to optimize a SNR based on electric signals obtained for a certain period, the signals having been photoelectrically converted by the optical detection unit.

### CROSS-REFERENCE TO RELATED APPLICATION

The present application is a Continuing application based on International Application PCT/JP2015/000612 filed on Feb. 10, 2015, the content of which is incorporated herein by reference.

### TECHNICAL FIELD

The present disclosure relates to a scanning apparatus and a method for controlling the same.

### BACKGROUND

As a conventional scanning endoscope apparatus, there has been known a system of displacing an emission end of an optical fiber extended through a scope so as to irradiate illumination light from the optical fiber toward an inspection site to scan the site, and generating an image by detecting light scatted at the site (see, for example, PTL 1).

The scanning endoscope apparatus of PTL 1 controls the irradiation timing of the illumination light, based on the detection timing of scattered light, so that the irradiation density of the illumination light is substantially fixed in the entire scanning area, to thereby eliminate useless irradiation with illumination light so as to obtain images of uniform brightness.

### CITATION LIST

### Patent Literature

PTL 1: JP2013-121455A

### SUMMARY

A scanning endoscope apparatus disclosed herein includes:

a light source;

an optical fiber that guides light emitted from the light source;

an actuator that deflects the light emitted from the optical fiber to repeatedly scan the light on an irradiation object;

a light detector controllable in multiplication factor, the light detector photoelectrically converting signal light obtained from the irradiation object irradiated with the light; and

a controller,

in which the controller controls the multiplication factor, based on electric signals photoelectrically converted by the light detector for a certain period, so as to optimize a signal-to-noise ratio.

The controller may control the multiplication factor so as to obtain a highest SNR for an electric signal with a minimum value, among the electric signals for the certain period.

The controller may control the multiplication factor so as to obtain a highest SNR for an electric signal with a maximum value, among the electric signals for the certain period.

The light detector may have an avalanche photodiode.

The light detector may have a photomultiplier tube.

The apparatus further includes an amplifier that amplifies the electric signals photoelectrically converted by the light detector, in which the controller may control a gain of the amplifier according to the multiplication factor of the light detector.

The controller may control the gain such that the product of the multiplication factor and the gain is obtained as a predetermined value.

Further, a method for controlling the disclosed scanning endoscope apparatus, includes:

deflecting, by an actuator, light emitted via an optical fiber from a light source so as to repeatedly scan an irradiation object;

photoelectrically converting, by a light detector controllable in multiplication factor, signal light obtained from the irradiation object irradiated with the light; and

controlling the multiplication factor, based on electric signals photoelectrically converted by the light detector for a certain period, so as to optimize a signal-to-noise ratio.

### BRIEF DESCRIPTION OF THE DRAWINGS

In the accompanying drawings:

FIG. 1 is a block diagram illustrating a schematic configuration of the disclosed scanning apparatus according to one embodiment;

FIG. 2 is an overview schematically illustrating the scope of FIG. 1;

FIG. 3 is a sectional view of the tip part of the scope of FIG. 2;

FIG. 4 is a flowchart for illustrating the main part of a method for controlling the scanning endoscope apparatus of FIG. 1; and

FIG. 5 shows a relation between the incident light quantity photoelectrically converted by the light detector of FIG. 1 and the SNR of an image.

### DETAILED DESCRIPTION

Hereinafter, an embodiment of the disclosed apparatus and method will be illustrated with reference to the accompanying drawings.

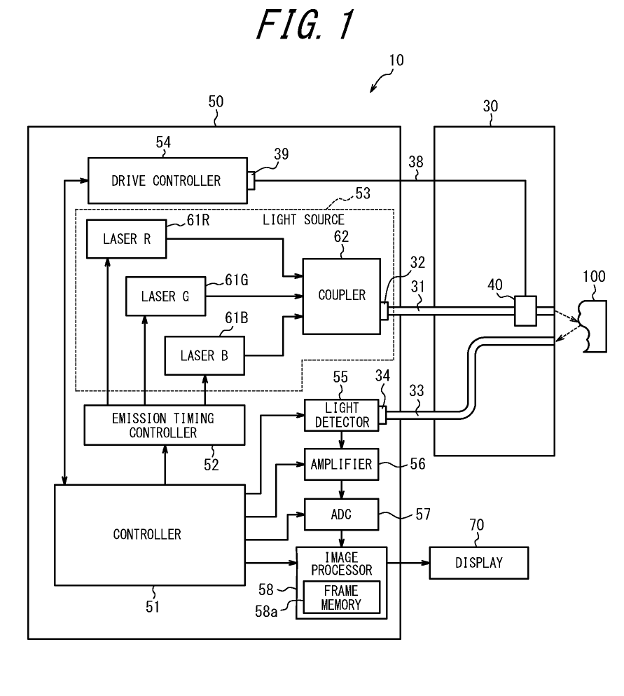

FIG. 1 is a block diagram showing a schematic configuration of a main part of the disclosed scanning endoscope according to one embodiment. The scanning endoscope apparatus 10 of this embodiment includes: a scope (endoscope) 30; a controller body (casing) 50; and a display 70. The controller body 50 includes: a controller 51 controlling the whole of the scanning endoscope apparatus 10; an emission timing controller 52; a light source 53; a drive controller 54; a light detector 55; an amplifier 56; an analog-digital converter (ADC) 57; and an image processor 58.

The light source 53 has lasers 61R, 61G, 61B and a coupler 62. The lasers 61R. 61G. 61B each emit red, green, and blue laser lights, respectively. The emission timing controller 52 controls the emission timings of the lasers 61R, 62G, 63B, under the control of the controller 51. The lasers 61R, 61G, 61B may use, for example, a diode pumped solid state laser (DPSS laser) or a laser diode. Laser lights (illumination lights) emitted from each of the lasers 61R, 62G, 63B are coaxially coupled by the coupler 62, to be incident on an illumination optical fiber 31. The coupler 62 is configured by including, for example, a dichroic prism. Without being limited to the aforementioned configuration, the light source 53 may include one laser or other plurality of light sources. Further, the light source 53 may be accommodated in a separate casing different from the controller body 50, the casing being connected with the controller body 50 via a signal line.

The illumination optical fiber 31 extends up to the tip of the scope 30. The illumination optical fiber 31 is coupled, via the incident end thereof, to a light input part 32 formed of, for example, a light connector. The light input part 32 is detachably coupled to the coupler 62 so as to cause illumination light from the light source 53 to be incident on the illumination optical fiber 31. The illumination optical fiber 31 has its emission end vibratorily supported by an actuator 40 to be described later. Illumination light incident on the illumination optical fiber 31 is guided through to the tip part of the scope 30, and emitted toward an object (irradiation object). At this time, the drive controller 54 supplies a predetermined drive signal to the actuator 40 so as to vibratorily drive the emission end of the illumination optical fiber 31. In this manner, illumination light emitted from the illumination optical fiber 31 is deflected, so that the object 100 is, for example, repeatedly scanned two-dimensionally with the illumination light in, for example, a known mode such as spiral scan or raster scan. Signal light such as reflected light, scattered light, and fluorescence to be obtained from the object 100 irradiated with illumination light is caused to be incident on the tip face of a detection optical fiber bundle 33 including a multi-mode fiber extending within the scope 30, so as to guided to the controller body 50.

The detection optical fiber bundle 33 is detachably coupled to the light detector 55 via a light connector 34, and guides signal light from the object 100 to the light detector 55. The light detector 55 receives signal light guided through the detection optical fiber bundle 33 and converts the signal light into an electric signal according to the color of the illumination light. The analog electric signal output from the light detector 55 is amplified by the amplifier 56 and converted by the ADC 57 into a digital signal, before being input to the image processor 58.

The controller 51 refers to information such as amplitude and phase of the drive signal supplied from the drive controller 54 to the actuator 40 so as to calculate information on the scanned position on the scanning locus of illumination light, and supplies the information thus calculated to the image processor 58. The image processor 58 has a frame memory 58a, and sequentially stores, in the frame memory 58a, electric signals (pixel data) on the object 100, output from the ADC 57 based on the scanned position information from the controller 51. Then, the image processor 58 subjects the image data stored in the frame memory 58a to predetermined image processing, so as to generate an image of the object 100 and display the image on the display 70. Here, the frame memory 58a may be incorporated in the controller 51, or may be disposed as an external memory.

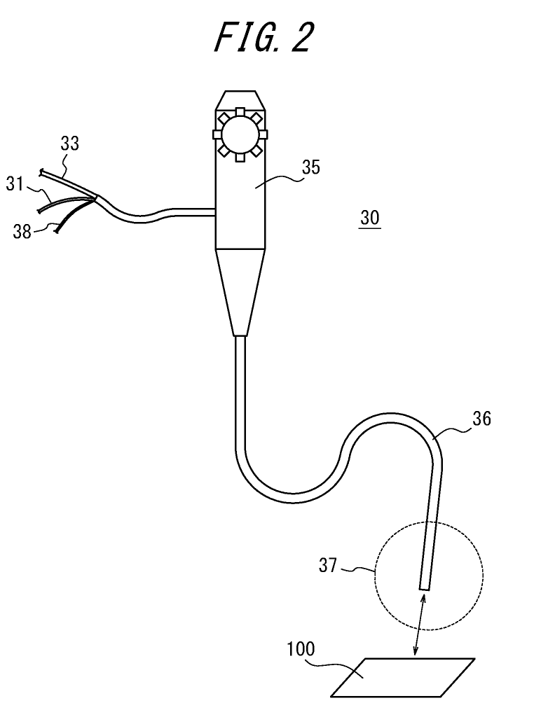

FIG. 2 is an overview schematically illustrating the scope 30. The scope 30 includes an operation portion 35 and an insertion portion 36. The illumination optical fiber 31 and the detection optical fiber bundle 33 are installed as being extended from the operation portion 35 to a tip part 37 (which is indicated by the broken line of FIG. 2) of the insertion portion 36 and each detachably connected to the controller body 50. The scope 30 also includes a wiring cable 38 which is connected to the actuator 40 and extends from the insertion portion 36 via the operation portion 35. The wiring cable 38 is detachably connected to the drive controller 54 via a connection connector 39, as shown in FIG. 1. Here, the insertion portion 36 is configured flexible to bend in part except for the tip part 37, the tip part 37 being formed as a hard part that do not bend.

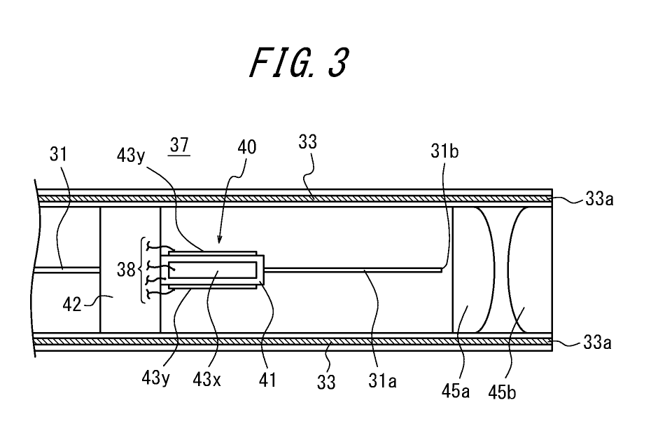

FIG. 3 is an enlarged sectional view of the tip part 37 of the scope 30 of FIG. 2. The tip part 37 has the actuator 40 and an illumination optical system 45 installed therein. FIG. 3 illustrates a case where the illumination optical system 45 is formed of two projection lenses 45a, 45b. The actuator 40 includes a ferrule 41 that supports an emission end face 31a of the illumination optical fiber 31 passing therethrough. The illumination optical fiber 31 is fixedly adhered to the ferrule 41. The ferrule 41 is coupled to a supporting part 42 at an end opposite to the emission end face 31b so as to be oscillatably cantilevered by the supporting part 42. The illumination optical fiber 31 penetrates through the supporting part 42 to be extended.

The ferrule 41 is formed of metal such as nickel. The ferrule 41 may be formed in an arbitrary outer shape, such as a rectangular column shape or a cylinder shape. The ferrule 41 has piezoelectric elements 43x and 43y mounted thereon, the piezoelectric elements 43x and 43y opposing to each other in the x-direction and in the y-direction, respectively, the x-direction and the y-direction being mutually orthogonal to each other in a plane perpendicular to the z-direction parallel to the optical axis direction of the illumination optical fiber 31. FIG. 3 shows only one piezoelectric element 43x. The piezoelectric elements 43x and 43y are each in a rectangular shape elongated in the z-direction. The piezoelectric elements 43x and 43y each have electrodes formed on both faces in the thickness direction, and are configured to extend and contract in the z-direction when applied with a voltage in the thickness direction via the opposing electrodes.

The piezoelectric elements 43x and 43y are each adhered to the ferrule 41 via one electrode surface while having the other electrode surface connected to the corresponding wiring cable 38. Similarly, the ferrule 41 serving as a common electrode of the piezoelectric elements 43x and 43y is connected to the corresponding wiring cable 38. The two opposing piezoelectric elements 43x in the x-direction are applied with an alternating voltage of the same phase from the drive controller 54 of FIG. 1 via the corresponding wiring cable 38. Similarly, the two opposing piezoelectric elements 43y in the y-direction are applied with an alternating voltage of the same phase from the drive controller 54 via the corresponding wiring cable 38.

With this configuration, one of the two piezoelectric elements 43x extends while the other contracts, to generate bending vibration in the x-direction in the ferrule 41. Similarly, one of the two piezoelectric elements 43y extends while the other contracts, to generate bending vibration in the y-direction in the ferrule 41. As a result, the x-direction vibration and the v-direction vibration of the ferrule 41 are combined, so that the ferrule 41 is deflected integrally with the emission end 31a of the illumination optical fiber 31. Accordingly, illumination light may be caused to incident on the illumination optical fiber 31, to thereby allow the object 100 to be two-dimensionally scanned with the illumination light emitted from the emission end face 31b.

The detection optical fiber bundle 33 passes through the outer periphery of the insertion portion 36 to extend up to the tip of the tip part 37. The detection optical fiber bundle 33 may have a detection lens (not shown) disposed at the tip part 33a of each fiber.

The projection lenses is disposed at the extreme tip of the tip part 37. The projection lenses 45a, 45b are configured to converge, onto a predetermined focal position, laser light emitted from the emission end face 31b of the illumination optical fiber 31. When the detection lens is disposed at the tip part 33a of the detection optical fiber bundle 33, the detection lens is arranged so as to take in, as signal light, light resulting from laser light irradiated onto the object 100 and reflected, scattered, and refracted by the object 100 (light that has been interacted with the object 100) or fluorescence, so as to have the light converged and coupled to the detection optical fiber bundle 33. The illumination optical system 45 may be formed of one lens or three or more lenses, without being limited to the two projection lenses 45a, 45b.

In this embodiment, the light detector 55 is configured with, for example, an avalanche photodiode or a photomultiplier tube, whose multiplication factor may be controlled by the controller 51. The amplifier 56 is configured such that the gain thereof may be controlled by the controller 51. The controller 51 controls the multiplication factor of the light detector 55, based on the electric signals for the past certain period (electric signals for the preceding one frame in this embodiment) stored in the frame memory 58a, such that the image in the next frame will have an optimum SNR. The controller 51 also controls the gain of the amplifier 56 according to the control of the multiplication factor of the light detector 55, such that the product of the multiplication factor of the light detector 55 and the gain of the amplifier 56 will be a predetermined value.

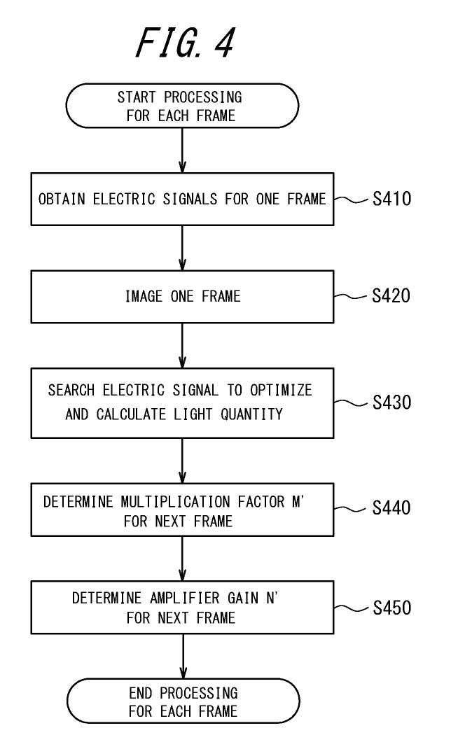

FIG. 4 is a flowchart for illustrating the main part a method for controlling the scanning endoscope apparatus of this embodiment, illustrating the process for each frame. First, the controller 51 controls the image processor 58 to obtain, in the frame memory 58a, electric signals for one frame (Step S410). The controller 51 then executes an imaging process for one frame (Step S420). In the imaging process for one frame, the controller 51 controls the image processor 58 to obtain electric signals for one frame, subjects the electric signals thus obtained to predetermined image processing (such as, for example, γ correction, interpolation, color balance adjustment, and structure emphasis) to generate an image of one frame, and displays the image thus generated on the display 70.

The controller 51 searches for an electric signal to serve as a reference to optimize the SNR, from electric signals for one frame stored in the frame memory 58a, after the process of Step S420 or in parallel with the process of Step S420, and obtains the incident light quantity thereof (Step S430). Here, the electric signal to serve as a reference to optimize the SNR may be, for example, the one having the minimum value or the maximum value, among the electric signals for one frame. Either the minimum value or the maximum value may be searched; one of them may always be searched in a fixed manner or the user may selectively specify which of the values to search. The incident light quantity is calculated based on, for example, the following equation. Without being limited to the following equation, the incident light quantity may be calculated based on electric signals by using a function, or may be obtained from a lookup table of the electric signal and the incident light quantity.

Electric Signal [V]=Incident Light Quantity [W]×Sensitivity of Light Detector [A/W]×Multiplication Factor M×Current Voltage Conversion Rate [V/A] of Light Detector×Gain N of Amplifier

Next, the controller 51 determines, based on the incident light quantity obtained in Step S430, the multiplication factor M′ of the light detector 55 for use in obtaining the electric signal for the next frame, and controls the multiplication factor M of the light detector 55 to the multiplication factor M′ thus determined (Step S440).

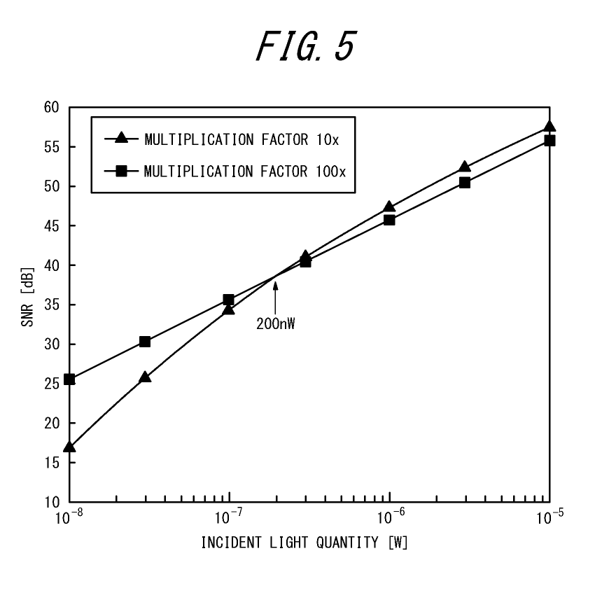

Here, the incident light quantity to be photoelectrically converted by the light detector 55 and the SNR of the image have an exemplary relation shown in FIG. 5, according to the properties of the photoelectric conversion elements constituting the light detector 55. In FIG. 5, the multiplication factors 10 and 100 have properties to be reversed in terms of the quality of the SNR across the incident light quantity of substantially 200 [nW]. That is, when the incident light quantity is less than 200 [nW], the SNR improves more with the multiplication factor 100 than with multiplication factor 10, whereas when the incident light quantity is larger than 200 [nW], the SNR improves more with the multiplication factor 10 than with the multiplication factor 100. Therefore, the controller 51 determines, depending on the obtained incident light quantity, the multiplication factor M′ which provides a higher SNR.

After that, the controller 51 determines, based on the multiplication factor M′ determined in Step S440, the gain N′ of the amplifier 56 for use in obtaining the electric signal for the next frame, and controls the gain N of the amplifier 56 to the gain N′ thus determined (Step S450). The gain N′ may be determined such that, for example, a gain G (G=M′×N′) represented by the product of the multiplication factor M′ and the gain N′ will be obtained as a predetermined value. Here, the gain G is a value, for example, for keeping constant the average luminance when imaged, and is determined according to the specification of the scanning endoscope apparatus 10.

The controller 51 executes the processing from Step S410 to S450 above for sequential frames.

In this embodiment, the multiplication factor of the light detector 55 may be controlled to a value which optimizes the SNR of the electric signal with the minimum value, to thereby generate an image having an optimized SNR in a dark region where noise is most often seen. Alternatively, the multiplication factor of the light detector 55 may be controlled to a value which optimizes the SNR of the electric signal with the maximum value, to thereby generate an image having an optimized SNR in a bright region which draws the most attention. Further, the gain of the amplifier 56 may be controlled according to the control of the multiplication factor such that the total gain G may be obtained as a predetermined value, to thereby prevent fluctuation in image luminance. Here, the image processor 58 may be used in place of the amplifier 56 to prevent fluctuation in image luminance. In this case, digital signals obtained from the ADC 57 may be controlled as being multiplied by the gain, which produces the same effect as obtained by controlling the gain of the amplification factor.

Here, the disclosed apparatus and method should not be limited to the aforementioned embodiment, and may be subjected to a number of modifications and alterations. For example, the multiplication factor may be controlled for every few frames, without being limited to the case of being sequentially controlled for each frame. Further, the electric signals of the object 100 obtained for the past certain period for controlling the multiplication factor could use electric signals for a plurality of the preceding frames, electric signals for one or a plurality of frames several frames before, or electric signals for less than one frame in the past, without being limited to the electric signals for the preceding one frame. Further, the electric signal to serve as a reference to optimize the SNR may be of an intermediate value (average value), without being limited to the electric signal of the minimum value or of the maximum value, among the electric signals obtained for the past certain period. This case allows for generating an image having the SNR optimized in a region of intermediate brightness. Further, the actuator 40 may employ an electromagnetic actuator which uses a coil and a permanent magnet, without being limited to the piezoelectric actuator, or may be configured to use MEMS mirror or the like to deflect illumination light emitted from the illumination optical fiber 31 to optically scan the illumination light without displacing the emission end of the illumination optical fiber 31. Further, the gain of the amplifier 56 may be kept constant irrespective of the control of the multiplication factor by the light detector 55, or may be varied at a predetermined ratio in accordance with the increase and decrease of the multiplication factor.

Further, the emission timing controller and the drive controller 54 may each be incorporated in part or in its entirety into the controller 51. Similarly, the amplifier 56, the ADC 57, and the image processor 58 may each be incorporated in part or in its entirety into the controller 51.

### REFERENCE SIGNS LIST

10 scanning endoscope apparatus
30 scope (endoscope)
31 illumination optical fiber
33 detection optical fiber bundle
40 actuator
51 controller
53 light source
55 light detector
56 amplifier
58 image processor
100 object (irradiation object)

1. A scanning endoscope apparatus, comprising:
a light source;
an optical fiber that guides light emitted from the light source;
an actuator that deflects the light emitted from the optical fiber to repeatedly scan the light on an irradiation object;
a light detector controllable in multiplication factor, the light detector photoelectrically converting signal light obtained from the irradiation object irradiated with the light; and
a controller,
wherein the controller controls the multiplication factor, based on electric signals photoelectrically converted by the light detector for a certain period, so as to optimize a signal-to-noise ratio.

2. The scanning endoscope apparatus according to claim 1, wherein the controller controls the multiplication factor so as to obtain a highest signal-to-noise ratio for an electric signal with a minimum value, among the electric signals for the certain period.

3. The scanning endoscope apparatus according to claim 1, wherein the controller controls the multiplication factor so as to obtain a highest signal-to-noise ratio for an electric signal with a maximum value, among the electric signals for the certain period.

4. The scanning endoscope apparatus according to claim 1, wherein the light detector has an avalanche photodiode.

5. The scanning endoscope apparatus according to claim 1, wherein the light detector has a photomultiplier tube.

6. The scanning endoscope apparatus according to claim 1, further comprising an amplifier that amplifies the electric signals photoelectrically converted by the light detector,
wherein the controller controls a gain of the amplifier according to the multiplication factor of the light detector.

7. The scanning endoscope apparatus according to claim 6, wherein the controller controls the gain such that the product of the multiplication factor and the gain is obtained as a predetermined value.

8. A method for controlling a scanning endoscope apparatus, comprising:
deflecting, by an actuator, light emitted via an optical fiber from a light source so as to repeatedly scan an irradiation object;
photoelectrically converting, by a light detector controllable in multiplication factor, signal light obtained from the irradiation object irradiated with the light; and
controlling the multiplication factor, based on electric signals photoelectrically converted by the light detector for a certain period, so as to optimize a signal-to-noise ratio.
